(19) United States
(12) Patent Application Publication
(10) Pub. No.: US 2021/0208266 A1
(43) Pub. Date: Jul. 8, 2021

(54) FUNDAMENTAL-AND-HARMONICS MULTI-FREQUENCY DOPPLER RADAR SYSTEM WITH RADAR MOTION CANCELLATION

(71) Applicant: POLYVALOR, LIMITED PARTNERSHI, Montreal (CA)

(72) Inventors: Zhu FANG, Montreal (CA); Kuangda WANG, Montreal (CA); Ke WU, Montreal (CA)

(21) Appl. No.: 17/056,647

(22) PCT Filed: May 21, 2019
(86) PCT No.: PCT/CA2019/050687

§ 371 Date: Nov. 18, 2020

Related U.S. Application Data
(60) Provisional application No. 62/673,460, filed on May 18, 2018.

(51) Int. Cl. G01S 13/34; G01S 13/56; G01S 13/526; A61B 5/0507

(52) U.S. Cl. CPC G01S 13/347; A61B 5/0507; G01S 13/526; G01S 13/56

(57) ABSTRACT

A Doppler radar system comprises a transceiver configured to concurrently transmit a first set of RF signals, having a first set of frequencies, towards target(s) in motion and a second set of RF signals, having a second set of frequencies, towards stationary reflector(s), concurrently receive a first set of reflected signals from the target(s) and a second set of reflected signals from the reflector(s), the first set of reflected signals modulated by motion of the target(s) and of a moving radar platform and the second set of reflected signals modulated by motion of the platform. The first and second sets of reflected signals are down-converted to generate a first and a second set of down-converted signals, which are demodulated to generate a first and a second set of demodulated signals, which are processed to obtain a third set of signals free of artifacts resulting from motion of the radar platform.

### CROSS REFERENCE TO RELATED APPLICATIONS

This patent application claims priority of U.S. provisional Application Ser. No. 62/673,460, filed on May 18, 2018, the entire contents of which are hereby incorporated by reference.

### TECHNICAL FIELD

The application relates generally to radar systems, and more particularly to a fundamental-and-harmonics multi-frequency (FHMF) Doppler radar system for vital signs detection, vibration detection, structural health monitoring, imaging, security, target classification, and motion and gesture detection with the presence of unwanted radar platform motion.

### BACKGROUND OF THE ART

Microwave Doppler radars have been used in a large number of applications, such as for the detection of vital signs and other physiological parameters. Their operation is based on the detection of a reflected signal that is modulated by the motion or displacement of a moving target, with the radar platform assumed to be stationary. Nonetheless, it is desirable in some applications to detect vital signs from a mobile platform. However, the unwanted motion of the radar platform introduces microwave signal path variations which also modulate the reflected signal. The combined motion along with related aliasing, phase distortion, and occurrence of null points in the received radar signal then make signal extraction challenging.

A number of solutions have been proposed to remove the influence of radar platform motion. For example, a bistatic radar structure with a sensor node receiver placed in the vicinity of the target was proposed. The bistatic configuration however requires complex data collection and the use of an over-the-air local-oscillator (LO) signal which may result in a poor signal-to-noise ratio (SNR). As an alternative method, an accelerometer was used to record the undesired radar platform motion, which was used for calibration in the radar signal processing. This method was proven to be effective only when the radar platform motion amplitude was small. Empirical mode decomposition (EMD) has also been applied to remove fidgeting interference. However, in the EMD method, intrinsic mode functions (IMFs) should be selected manually, which is not always possible.

Another solution was to use cameras to characterize the motion of the radar platform. However, using cameras is not always practically feasible. Moreover, harmonic tags and low intermediate frequency (IF) tags placed on stationary platform can also be introduced to extract the signal components due to unwanted radar platform motion. However, the power efficiency of the tags is usually low, so long-distance operation is impossible. In addition, it is difficult to use tags in the see-through-wall (STW) applications, where the targets are behind the wall. In other solutions, additional ultrasonic sensors were used in the STW applications to record the undesired radar platform motion signal. However, ultrasonic sensors usually have a limited range resolution and therefore make it difficult to detect relatively small radar platform motion.

Therefore, there is a need for an improved mobile Doppler radar that can eliminate the unwanted signal component generated by the motion of the radar platform.

### SUMMARY

In one aspect, there is provided a Doppler radar system comprising a transceiver mounted on a moving platform, the transceiver configured to concurrently transmit a first set of radio frequency (RF) signals and a second set of RF signals, the first set of signals having a first set of frequencies and transmitted towards one or more targets in motion, and the second set of signals having a second set of frequencies and transmitted towards one or more RF signal reflectors stationary in the coordinate system of the one or more targets, concurrently receive a first set of reflected signals and a second set of reflected signals, the first set of reflected signals received from the one or more targets and modulated by motion of the one or more targets and by motion of the radar platform, and the second set of reflected signals received from the one or more reflectors and modulated by motion of the radar platform, and down-convert the first set of reflected signals and the second set of reflected signals to generate a first set of down-converted signals and a second set of down-converted signals. The Doppler radar system comprises a processing unit configured to demodulate the first set of down-converted signals and the second set of down-converted signals to generate a first set of demodulated signals and a second set of demodulated signals.

In some embodiments, the processing unit is configured to process the first and second sets of demodulated signals to obtain a third set of signals free of artifacts resulting from motion of the radar platform.

In some embodiments, the processing unit is configured to subtract the second set of demodulated signals from the first set of demodulated signals to obtain the third set of signals.

In some embodiments, the processing unit is further configured to digitize and process the first set of down-converted signals and the second set of down-converted signals prior to demodulation thereof.

In some embodiments, the first set of signals comprise a first set of frequency components of T₁*f₀, T₂*f₀, . . . Tₖ*f₀ of a periodic oscillating signal, where T₁˜Tₖ are positive integers and f₀ is the fundamental frequency of the Fourier decomposition of the signal, and the second set of signals comprises a second set of frequency components of P₁*f₀, P₂*f₀, . . . Pₙ*f₀ of the periodic oscillating signal, where P₁˜Pₖ are positive integers.

In some embodiments, the transceiver comprises a first set of antennas configured to transmit the first set of signals and receive the first set of reflected signals, and a second set of antennas configured to transmit the second set of signals and receive the second set of reflected signals.

In some embodiments, the transceiver comprises a first set of transmitting antennas configured to transmit the first set of signals, a first set of receiving antennas configured to receive the first set of reflected signals, a second set of transmitting antennas configured to transmit the second set of signals, and a second set of receiving antennas configured to receive the second set of reflected signals.

In some embodiments, at least one of the antennas is a high front-to-back ratio (FBR) antenna.

In some embodiments, the first set of signals and the second set of signals are generated by converting a sum of one or more sinusoidal signals into a sum of harmonic components.

In some embodiments, the first set of signals and the second set of signals are generated from a combination of a plurality of outputs of a plurality of signal generators.

In some embodiments, the transceiver comprises a coherent multi low-intermediate frequency (IF) receiver configured to concurrently receive the first and second sets of reflected signals, the receiver comprising multiple coherent low-IF receiver chains in parallel.

In some embodiments, the transceiver comprises a single set of circuitries used for concurrently transmitting the first and second sets of signals and concurrently receiving the first and second sets of reflected signals.

In another aspect, there is provided a method for operating a Doppler radar system, the method comprising concurrently transmitting a first set of RF signals and a second set of RF signals, the first set of signals having a first set of frequencies and transmitted towards one or more targets in motion, and the second set of signals having a second set of frequencies and transmitted towards one or more RF signal reflectors stationary in the coordinate system of the one or more targets, concurrently receiving a first set of reflected signals and a second set of reflected signals, the first set of reflected signals received from the one or more targets and modulated by motion of the one or more targets and by motion of a moving radar platform, and the second set of reflected signals received from the one or more reflectors and modulated by motion of the radar platform, down-converting the first set of reflected signals and the second set of reflected signals to generate a first set of down-converted signals and a second set of down-converted signals, demodulating the first set of down-converted signals and the second set of down-converted signals to generate a first set of demodulated signals and a second set of demodulated signals, and processing the first and second sets of demodulated signals to obtain a third set of signals free of artifacts resulting from motion of the radar platform.

In some embodiments, processing the first and second sets of demodulated signals comprises subtracting the second set of demodulated signals from the first set of demodulated signals to obtain the third set of signals.

In some embodiments, the method further comprises digitizing and processing the first set of down-converted signals and the second set of down-converted signals prior to demodulation thereof.

In some embodiments, concurrently transmitting the first set of RF signals and the second set of RF signals comprises concurrently transmitting the first set of signals comprising a first set of frequency components of a periodic oscillating signal and the second set of signals comprising n a second set of frequency components of the periodic oscillating signal.

In some embodiments, the first set of signals and the second set of signals are generated by converting a sum of one or more sinusoidal signals into a sum of harmonic components.

In some embodiments, the first set of signals and the second set of signals are generated from a combination of a plurality of outputs of a plurality of signal generators.

In some embodiments, the first and second sets of reflected signals are concurrently received at a coherent multi low-intermediate frequency (IF) receiver comprising multiple coherent low-IF receiver chains in parallel.

In some embodiments, the first and second sets of signals are concurrently transmitted, and the first and second sets of reflected signals are concurrently received via a single set of circuitries.

In yet another aspect, there is provided a receiver for a Doppler radar system, the receiver comprising a first set of receiving antennas operating at a first set of frequencies and configured to receive a first set of RF signals, the first set of signals reflected from one or more targets in motion and having the first set of frequencies, a second set of receiving antennas operating at a second set of frequencies and configured to receive a second set of RF signals, the second set of signals reflected from one or more reflectors stationary in the coordinate system of the one or more targets, the second set of signals having the second set of frequencies, a first set of output ports and a second set of output ports, a first signal channel connected to the first set of receiving antennas and the first set of output ports and a second signal channel connected to the second set of receiving antennas and the second set of output ports, the first and second signal channels each comprising one or more signal paths, and pseudo-diplexer circuitries configured to selectively direct the first set of reflected signals over the first signal channel and the second set of reflected signals over the second signal channel.

### DESCRIPTION OF THE DRAWINGS

Reference is now made to the accompanying figures in which:

FIG. 1A is a block diagram of an FHMF Doppler radar system, in accordance with an illustrative embodiment;

FIG. 1B is a block diagram of an FHMF Doppler radar system, in accordance with another illustrative embodiment;

FIG. 2A is a detailed block diagram of the FHMF Doppler radar unit of FIG. 1A and FIG. 1B with a single receiver, in accordance with one embodiment;

FIG. 2B is a detailed block diagram of the FHMF Doppler radar unit of FIG. 1A and FIG. 1B with two receivers, in accordance with one embodiment;

FIG. 2C is a detailed block diagram of the FHMF Doppler radar unit of FIG. 1A and FIG. 1B with two receivers, in accordance with another embodiment;

FIG. 3A and FIG. 3B are plots, in the time-domain and the frequency-domain, respectively, of results measured using the FHMF Doppler radar system of FIG. 1A, when no Adaptive Noise Cancellation (ANC) technique is used;

FIG. 4A and FIG. 4B are plots, in the time-domain and the frequency-domain, respectively, of results measured using the FHMF Doppler radar system of FIG. 1A, when an ANC technique is used;

FIG. 5A and FIG. 5B are plots, in the time-domain and the frequency-domain, respectively, of results measured using the FHMF Doppler radar system of FIG. 1B, when no ANC technique is used;

FIG. 6A and FIG. 6B are plots, in the time-domain and the frequency-domain, respectively, of results measured using the FHMF Doppler radar system of FIG. 1B, when an ANC technique is used; and

FIG. 7 is a flowchart of an example method for operating a Doppler radar system, in accordance with one embodiment.

It will be noted that throughout the appended drawings, like features are identified by like reference numerals.

### DETAILED DESCRIPTION

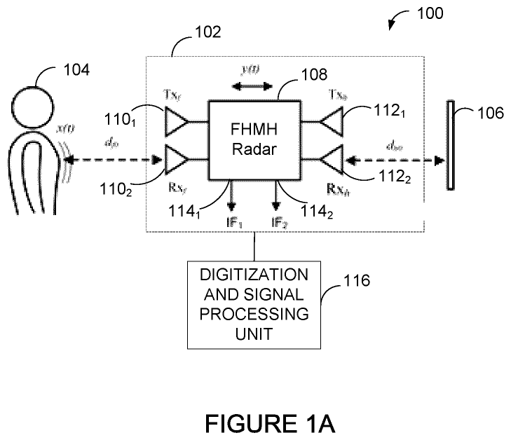

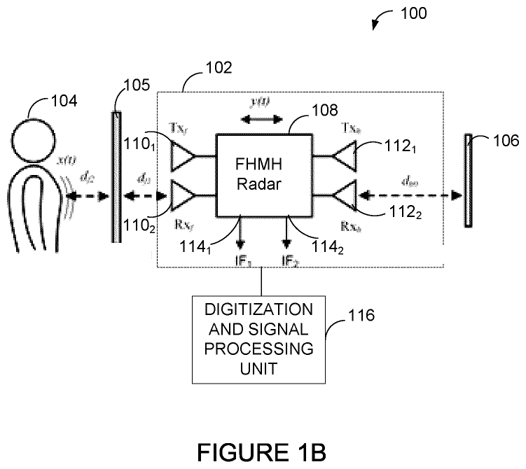

Referring to FIG. 1A, an FHMF Doppler radar system 100, in accordance with one embodiment, will now be described. The FHMF Doppler radar system 100 is configured for non-contact motion, gesture and vital signs detection with radar platform motion cancellation. The radar system 100 comprises an FHMF Doppler radar unit 102, which is configured to monitor the motion, gesture and vital signs (e.g. the respiration and/or heartbeat of a living subject) of a target 104 while removing the influence of unwanted motion of the radar system 100 on a moving platform. The FHMF Doppler radar unit 102 may be provided as a mobile device (e.g. carried by an operator or mounted on an unmanned vehicle) that enables flexible and continuous operation. In particular, measurements may be acquired, and vital signs detection may be performed on the go.

The radar system 100 may be used for a number of applications including, but not limited to, life health monitoring, house security (e.g. anti-thief) monitoring, structural health monitoring, vibration detection and imaging applications. As shown in FIG. 1B, the radar system 100 may also be used for mobile STW applications including, but not limited to, rescue in disaster site and security applications. For example, in search and rescue applications, the radar system 100 may be used to detect living subjects buried under ruins or hidden behind various barriers. In particular, the system 100 may be used to detect and monitor the vital signs of the target 104 with an obstacle (e.g. a wall or the like) 105 positioned between the FHMF Doppler radar unit 102 and the target 104. STW vital signs detection from a mobile platform can then be performed using the radar system 100 without the need of additional devices for radar platform motion calibration, such as ultrasonic sensors, accelerometer and the like.

As shown in FIG. 1A and FIG. 1B, a reflector 106 is positioned at a nominal distance dₕ₀ from the FHMF Doppler radar unit 102 and is used as a stationary reference. The reflector 106 may be any object in the surrounding environment (e.g. a metal plate, an obstacle, a wall, ceiling, floor, or the like) that can reflect RF signals back to the radar unit 102. The reflector 106 is stationary in the coordinate system of the target 104. In one embodiment, the reflector 106 is stationary whereas the target 104 may be in motion. In another embodiment, the reflector 106 and the target 104 are both in a non-stationary reference system. It should be understood that although a single target 104 and a single reflector 106 are illustrated and described herein, more than one target as in 104 and more than one reflector as in 106 may apply.

The FHMF Doppler radar system 100 further comprises an FHMF radar transceiver 108, which is provided in the FHMF Doppler radar unit 102. In one embodiment, the FHMF radar transceiver 108 has a high front-to-back ratio (FBR) transmitting fundamental antenna (Tx_{f}) 110₁, a high FBR receiving fundamental antenna (Rx_{f}) 110₂, a high FBR transmitting harmonic antenna (Txₕ) 121, a high FBR receiving harmonic antenna (Rxₕ) 112₂, a first low-IF output port (IF₁) 114₁ and a second low-IF output port (IF₂) 114₂. As used herein, the term “fundamental antenna” refers to an antenna operating at the fundamental frequency and the term “harmonic antenna” refers to an antenna operating at the second harmonic frequency. The FHMF radar transceiver 108 may be mounted on a mobile platform (not shown).

In one embodiment, separate fundamental antennas 110₁, 110₂ are used for transmitting and receiving the fundamental frequency component and separate harmonic antennas 112₁, 112₂ are used for transmitting and receiving the second harmonic frequency component. It should however be understood that a single fundamental antenna can replace the pair of fundamental antennas 110₁, 110₂ for transmitting and receiving the fundamental frequency component concurrently. Similarly, a single harmonic antenna can replace the pair of harmonic antennas 112₁, 112₂ for transmitting and receiving the second harmonic frequency component concurrently. It should also be understood that the transceiver 108 may comprise a set of transmitting fundamental antennas, a set of receiving fundamental antennas, a set of transmitting harmonic antennas, and a set of receiving harmonic antennas. The transceiver 108 may accordingly comprise a first set of low-IF output port as in 114₁ and a second set of low-IF output port as in 114₂.

In one embodiment, the transmitting fundamental antenna 110₁ is identical to the receiving fundamental antenna 110₂ and the transmitting harmonic antenna 112₁ is identical to the receiving harmonic antenna 112₂. In one embodiment, the fundamental and harmonic antennas 110₁, 110₂, 112₁, and 112₂ are aperture-coupled patch antennas. It should however be understood that other embodiments may apply. It should also be understood that the sizes of the antennas 110₁, 110₂, 112₁, and 112₂ may be different due to their different operating frequencies.

The pair of fundamental antennas 110₁, 110₂ and the pair of harmonic antennas 112₁, 112₂ are provided in opposite directions. In particular, the fundamental antennas 110₁, 110₂ are directed towards the target 104, which is positioned at a nominal distance d_{f0} from the antennas 110₁, 110₂, and the harmonic antennas 112₁, 112₂ are directed towards the reflector 106, which is positioned at the nominal distance dₕ₀ from the antennas 112₁, 112₂. Thus, the fundamental antennas 110₁, 110₂ only see (i.e. exchange signal(s) with) the target 104 while the harmonic antennas 112₁, 112₂ only see the reflector 106. However, due to the limited FBR of the real-world antennas, the fundamental and harmonic antennas can also slightly see in their backward directions. In other embodiments, the pair of fundamental antennas 110₁, 110₂ and the pair of harmonic antennas 112₁, 112₂ are provided not in opposite directions, but in other relative angles. In some embodiments, the relative angle is tunable during the operation of the radar system.

As will be discussed further below, the FHMF Doppler radar unit 102 concurrently transmits, into a region under observation and via the transmitting antennas 1101 and 112₁, the fundamental component (f₀) and the second harmonic component (2f₀) of the Fourier decomposition of a periodic oscillating signal generated by a suitable source (e.g. an electronic oscillator, not shown). It should however be understood that, in some embodiments, the FHMF Doppler radar unit 102 may be configured to concurrently transmit other signal components, namely x*f₀ and y*f₀, where x and y are integers and x≠y. In addition, the FHMF Doppler radar unit 102 may be configured for operation with more than two (2) harmonic components of the Fourier decomposition of the periodic signal.

The signal components are transmitted towards different directions, with the fundamental signal component being transmitted by the fundamental transmitting antenna 110₁ towards the target 104 and the harmonic signal component being transmitted by the harmonic transmitting antenna 112₁ towards the reflector 106. The transmitted fundamental signal component is then reflected by the target 104 (and subsequently received at the fundamental receiving antenna 110₂) and the harmonic signal component is reflected by the reflector 106 (and subsequently received at the harmonic receiving antenna 112₂). As governed by the Doppler principle, the target 104 and the reflector 106 each changes the phase and frequency of the reflected signals in accordance the velocity of the target 104 and of the radar platform. By transmitting the fundamental and the second harmonic signal components towards different directions, the reflected signals can then carry different messages. The reflected fundamental signal component is indeed modulated by both motion of the target 104 and motion of the platform the FHMF radar transceiver 108 is positioned on. In contrast, the reflected harmonic signal component is only modulated by motion of the FHMF Doppler radar unit 102 and can be used as a reference signal to detect and remove the unwanted radar platform motion. As such, the reflector 106 is sufficient to extract and separate the radar platform motion.

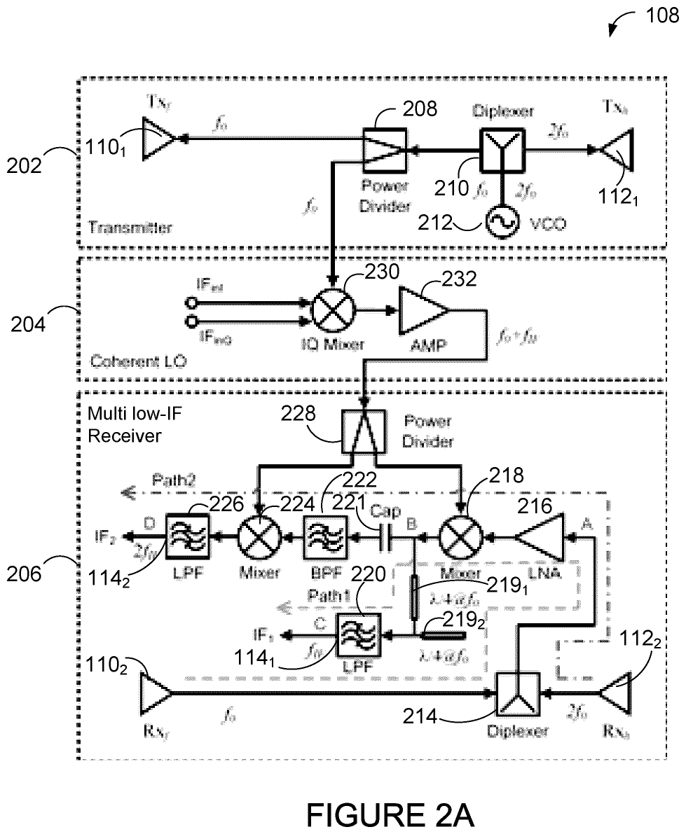

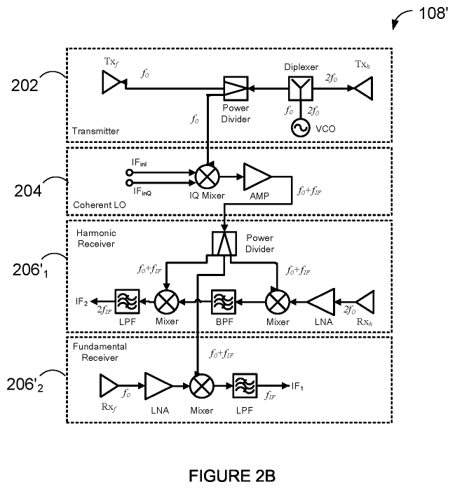

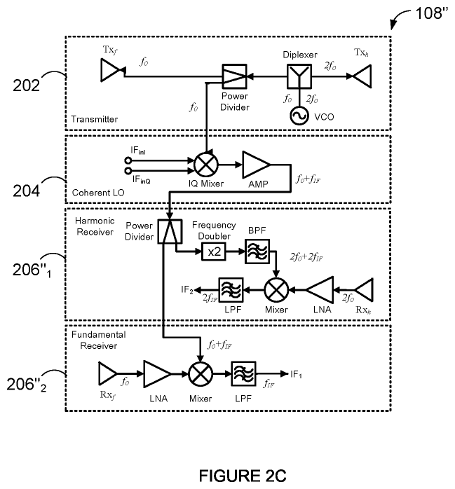

Referring now to FIG. 2A in addition to FIG. 1A and FIG. 1B, in one embodiment, the FHMF radar transceiver 108 comprises a transmitter 202, a coherent LO unit 204, and a multi low-IF receiver 206.

The transmitter 202 comprises the transmitting fundamental antenna 110₁, the transmitting harmonic antenna 112₁, a power divider 208, a diplexer 210, and an oscillator 212. In one embodiment, the oscillator 212 is a voltage-controlled oscillator (VCO). As discussed above, using the transmitter 202, the FHMF radar transceiver 108 can concurrently transmit both the fundamental signal component (f₀) and the inherent second harmonic frequency component (2f₀) of the signal output by the oscillator 212, with f₀ being the fundamental oscillation frequency of the signal output by the oscillator 212. The output of the oscillator 212 indeed inevitably contains harmonics, as will be understood by a person skilled in the art, and the proposed FHMF radar transceiver 108 utilizes both the fundamental and the second harmonic frequency components of the signal output by the oscillator 212. In one embodiment, by using the inherent second harmonic of the oscillator 212, only one oscillator 212 is required for a multi-frequency radar system 100, thereby reducing power consumption and cost. In another embodiment, the oscillator 212 is designed to boost the harmonic signal component amplitude compared to normal oscillator or VCO designs, thereby increasing power efficiency.

The two frequency components (f₀ and 2f₀) of the RF signal output by the oscillator 212 are separated in the transmitter 202 using the diplexer 210 and fed to the transmitting antennas 110₁ and 112₁, respectively. In some embodiments, the transmitter 202 works with more than two frequency components at more than two frequencies, and diplexer 210 is replaced with a multiplexer accordingly. As discussed above, the fundamental signal component is transmitted towards the target 104 by the fundamental transmitting antenna 110₁, which operates at f₀, while the harmonic signal component is transmitted towards the stationary reflector 106 by the harmonic transmitting antenna 112₁, which operates at 2f₀. The transmitted fundamental signal component can be expressed as:

Tx_{f}(t)=A_{tf} cos[2πf₀t+ϕ(t)] (1)

and the transmitted second harmonic signal component can be expressed as:

Txₕ(t)=Aₜₕ cos[2π(2f₀)t+2ϕ(t)] (2)

where f₀ is the fundamental oscillation frequency of the signal output by the oscillator 212, t is the elapsed time, and ϕ(t) is the phase noise of the fundamental signal component. The terms A_{tf} and Aₜₕ represent the amplitudes of the transmitted fundamental and harmonic signal components, respectively.

The transmitted fundamental signal component is then reflected by the target 104 and the harmonic signal component is reflected by the reflector 106. As discussed above, the reflected signals from the target 104 and the reflector 106 are then respectively received by the receiving fundamental antenna 110₂ and the receiving harmonic antenna 112₂. The reflected fundamental signal component from the target 104 can be approximated as:

$${Rx}_{f} ( t ) \approx {A}_{rf} cos [ 2 π {f}_{0} t - {θ}_{f} - \frac{4 π ( x ( t ) + y ( t ) )}{λ} + φ ( t - \frac{2 {d}_{f 0}}{c} ) ] \qquad ( 3 )$$

and the reflected harmonic signal component from the reflector 106 can be approximated as:

$${Rx}_{h} ( t ) \approx {A}_{rh} cos [ 2 π ( 2 {f}_{0} ) t - {θ}_{h} + \frac{4 π y ( t )}{λ / 2} + 2 φ ( t - \frac{2 {d}_{h 0}}{c} ) ] \qquad ( 4 )$$

where x(t) is the time varying target motion, y(t) is the time varying radar platform motion, λ is the free space wavelength of sinusoidal RF signal at frequency f₀, c is the signals' propagation velocity in air, and the terms A_{rf} and Aᵣₕ represent the amplitudes of the received signals at f₀ and 2f₀, respectively. The constant phase shifts θ_{f} and θₕ, respectively, are given as:

$$\begin{matrix}{θ}_{f} = \frac{4 π {d}_{0}}{λ} + {θ}_{f 0} & ( 5 ) \\ and & \\ {θ}_{h} = \frac{8 π {d}_{h 0}}{λ} + {θ}_{h 0} & ( 6 )\end{matrix}$$

where θ_{f0} represents the constant phase shift due to the reflection at the surface of the target 104 and θₕ₀ represents the constant phase shift due to the reflection at the surface of the reflector 106.

Still referring to FIG. 2A in addition to FIG. 1A and FIG. 1B, in one embodiment, the receiver 206 has a coherent multi low-IF architecture and can be seen as two coherent low-IF receiver chains provided in parallel, which allows the reflected fundamental and second harmonic signal components to be received concurrently without aliasing. In particular, the architecture of the receiver 206 uses a single receiver chain for full phase recovery, resulting in a simple architecture. Still, it should be understood that two separate receivers operating at two different frequencies (f₀ and 2f₀, respectively) may be used to achieve the same function as the receiver 206. This is illustrated in FIG. 2B and FIG. 2C. FIG. 2B shows a FHMF radar transceiver 108′ that comprises the transmitter 202, the coherent LO unit 204, a harmonic receiver 206′₁ and a fundamental receiver 206′₂. FIG. 2C shows a FHMF radar transceiver 108″ that comprises the transmitter 202, the coherent LO unit 204, a harmonic receiver 206″₁ and a fundamental receiver 206″₂. The harmonic receivers 206′₁ and 206″₁ are configured to receive the harmonic signal component while the fundamental receivers 206′₂ and 206″₂ are configured to receive the fundamental signal component. These embodiments however result in larger size, higher power consumption, and higher cost compared to the embodiment of FIG. 2A, which is therefore preferred.

Referring back to FIG. 2A in addition to FIG. 1A and FIG. 1B, in the embodiment where a multi low-IF receiver architecture is used, the receiver 206 comprises the receiving fundamental antenna 110₂ operating at f₀, the receiving harmonic antenna 112₂ operating at 2f₀, a diplexer 214, a low noise amplifier (LNA) 216, a first mixer—218, a high impedance transmission line 2191, a shunt open stub 219₂, a first low-pass filter (LPF) 220, a capacitor 221, a bandpass filter (BPF) 222, a second mixer 224, a second LPF 226, a power divider 228, the first low-IF output port (IF₁) 1141, and the second low-IF output port (IF₂) 114₂.

As will be discussed further below, the fundamental signal component received by the receiving fundamental antenna 110₂ is passed to the IF₁ port 114₁ through a first signal channel or path (labelled Path1 in FIG. 2A) while the harmonic signal component received by the receiving harmonic antenna 112₂ is passed to the IF₂ port 114₂ through a second signal channel or path (labelled Path2 in FIG. 2A). Each of the first and second signal channels may comprise one or more signal paths. The signal provided to the IF₁ port 1141, referred to herein as the IF₁ signal, and the signal provided to the IF₂ port 114₂, referred to herein as the IF₂ signal, may be recorded using any suitable means (e.g. an oscilloscope or a digitizer) then processed by a suitable processing device. The processing device can comprise, for example, any type of general-purpose microprocessor or microcontroller, a digital signal processor (DSP), a central processing unit (CPU), an application-specific integrated circuit (ASIC), a field programmable gate array (FPGA), a reconfigurable processor, other suitably programmed or programmable logic circuits, or any combination thereof.

The IF₁ and IF₂ signals are centered at f_{IF} and 2f_{IF}, respectively, where f_{IF} is the low-IF frequency. In this manner and as will be discussed further below, the reflected fundamental and second harmonic signal components can be received concurrently by a single receiver (receiver 206) without aliasing.

The fundamental signal component received at the receiving fundamental antenna 110₂ and the harmonic signal component received at the receiving harmonic antenna 112₂ are combined by the diplexer 214. The combined signal is output by the diplexer 214 at point A and can be expressed as:

R_{A}(t)=Rx_{f}(t)+Rxₕ(t) (7)

After being amplified by the LNA 216, the combined signal R_{A}(t) is mixed with a coherent low-IF LO signal at the mixer 218 to obtain a signal R_{B}(t) at point B. In particular, the coherent low-IF LO signal (centered at f₀+f_{IF}) is generated in the coherent LO unit 204 by mixing at an in-phase/quadrature (I/Q) mixer (also referred to as an image-reject up converter) 230 the fundamental signal component of the oscillator or VCO, which is identical to the transmitted fundamental RF signal, and an input quadrature low-IF signal (labelled IF_{inI} and IF_{inQ} FIG. 2A) and amplifying the resulting signal at an amplifier 232. In one embodiment, because the transmitted RF signal is used to construct a coherent low-IF LO signal that is highly correlated in phase noise to that of the transmitted signal, the coherent low-IF architecture described herein has the benefit of range correlation.

In one embodiment, the input quadrature low-IF signal is illustratively provided at I/Q outputs of a vector signal generator (not shown) and the coherent low-IF LO signal L_{O}(t) that is output by the coherent LO unit 204 can be represented as:

Lo(t)=cos[2π(f₀+f_{IF})t+ϕ(t)] (8)

The coherent low-IF LO signal L_{O}(t) is then divided at a power divider 228 prior to being provided as an input to the mixer 218.

The signal R_{B}(t) obtained at point B (i.e. at the output of the mixer 218) can then be written as:

$${R}_{B} ( t ) \approx {A}_{Bf} cos [ 2 π {f}_{IF} t + {θ}_{f} + \frac{4 π ( x ( t ) + y ( t ) )}{λ} + {Δφ}_{1} ( t ) ] + {A}_{Bh} cos [ 2 π ( {f}_{0} - {f}_{IF} ) t - {θ}_{h} + \frac{8 π y ( t )}{λ} + 2 φ ( t - \frac{2 {d}_{h 0}}{c} ) - φ ( t ) ] \qquad ( 9 )$$

where the terms A_{Bf} and A_{Bh} are the amplitudes of the signals centered at f_{IF} and f₀-f_{IF}, respectively, and Δϕ₁(t) is the residual phase noise at f₀ and is given by:

$${Δφ}_{1} ( t ) = φ ( t ) - φ ( t - \frac{2 {d}_{f 0}}{c} ) \qquad ( 10 )$$

Comparing equations (7) and (9), it can be seen that the first term in equation (9) is the down-converted fundamental signal component, which is centered at f_{IF}, while the second term in equation (9) is the down-converted harmonic signal component, which is centered at f₀-f_{F}. For simplicity, the first term of the R_{B}(t) signal is represented as R_{B1}(t) and the second term of the R_{B1}(t) signal is represented as R_{B2}(t). The capacitor 221, whose function is similar to that of a Direct Current (DC) block, is then used to block R_{B1}(t) in Path2 such that the R_{B1}(t) signal can only be passed to IF₁ port 114₁ through the first path. In one embodiment, because f_{IF} is a low frequency (typically in the kilohertz up to low megahertz range), the capacitor 221 is chosen so as to have a high-pass characteristic sufficient to block the R_{B1}(t) signal in the second path. On the other hand, a series λ_{g}/4 high impedance transmission line 219₁ (where λ_{g} is the guided wavelength on a substrate at f₀-f_{IF}) and a shunt λ_{g}/4 open stub 219₂ are introduced in the first path to block the R_{B2}(t) signal.

As will be understood by a person skilled in the art, point B can be viewed as an open circuit in the first path for the signals whose frequencies are located at the vicinity of f₀-f_{IF} due to the series λ_{g}/4 high impedance transmission line 219₁ and the shunt λ_{g}/4 open stub 219₂. Therefore, the R₂(t) signal can only be passed to the IF₂ port 114₂ through the second path. It should be understood that the capacitor 221 in the second path has no influence on the R₂(t) signal and the two λ_{g}/4 high impedance transmission lines 219₁, 219₂ in the first path has no influence on the R_{B1}(t) signal. The circuit which consists of the capacitor 221 and the two λ₉/4 high impedance transmission lines 219₁, 219₂ is similar to a frequency diplexer with point B as the sum port and may therefore be referred to herein as a “pseudo-diplexer”. In this manner, the reflected fundamental and second harmonic signal components are separated effectively using a single receiver 206 having two outputs 114₁, 114₂.

In the first path, the first LPF 220 is used to filter the R_{B1}(t) signal and the output of the LPF 220 obtained at point C (i.e. the IF₁ signal provided to the IF₁ port 114₁) can be expressed as:

$${IF}_{1} ( t ) \approx {A}_{Cf} cos [ 2 π {f}_{IF} t + {θ}_{f} + \frac{4 π ( x ( t ) + y ( t ) )}{λ} + {Δφ}_{1} ( t ) ] \qquad ( 11 )$$

where A_{Cf} represents the amplitude of the IF₁ signal.

In the second path, the BPF 222 (having a center frequency of f₀-f_{IF}) is used to suppress spurious signals (other than the one specified in equation (9)) at the output of the mixer 218. The signal output by the BPF 222 is then mixed at the mixer 224 with the signal output by the power divider 228 (i.e. with the coherent low-IF LO signal). After the second stage of down-conversion at the mixer 224 and low-pass filtering at the LPF 226, the signal obtained at point D (i.e. the IF₂ signal provided to the IF₂ port 114₂) is then given as:

$${IF}_{2} ( t ) \approx {A}_{Dh} cos [ 4 π {f}_{IF} t + {θ}_{h} - \frac{8 π y ( t )}{λ} + 2 {Δφ}_{2} ( t ) ] \qquad ( 12 )$$

where A_{Dh} is the amplitude of the IF₂ signal and Δϕ₂(t) is the residual phase noise at 2f₀ and is given by:

$${Δφ}_{2} ( t ) = φ ( t ) - φ ( t - \frac{2 {d}_{2}}{c} ) \qquad ( 13 )$$

From equations (11) and (12), it can be seen that the IF₁ signal is centered at f_{IF} and the IF₂ signal is centered at 2f_{IF}. In order to I/Q down-convert the IF₁ signal, the signal in equation (11) is first recorded and then multiplied by 2 exp(−j2πf_{IF}t) in the processing device, as follows:

2IF₁(t)exp(−j2πf_{IF}t)=A_{Cf} cos(φ_{f}(t))+jA_{Cf} sin(φ_{f}(t))+A_{Cf} cos(4πf_{IF}t+φ_{f}(t))−jA_{Cf} sin(4πf_{IF}t+φ_{f}(t)) (14)

where:

$${φ}_{f} ( t ) = {θ}_{f} + \frac{4 π ( x ( t ) + y ( t ) )}{λ} + {Δφ}_{1} ( t ) \qquad ( 15 )$$

Removing the 2f_{IF} components in equation (14) using a digital low-pass filter, the down-converted IF₁ signals in the I channel and the Q channel can be respectively represented as:

B_{fI}(t)=A_{Cf} cos(φ_{f}(t)) (16)

B_{fQ}(t)=A_{Cf} sin(φ_{f}(t)) (17)

Similarly, the IF₂ signal is recorded and multiplied by 2 exp(−j4πf_{IF}t) in the processing device. Removing the 4f_{IF} components using a digital low-pass filter, the down-converted IF₂ signals in the I channel and the Q channel can be respectively represented as:

$$\begin{matrix}{B}_{hI} ( t ) = {A}_{Df} cos ( {ϕ}_{h} ( t ) ) & ( 18 ) \\ {B}_{hQ} ( t ) = {A}_{Dh} sin ( {ϕ}_{h} ( t ) ) & ( 19 ) \\ where & \\ {ϕ}_{h} ( t ) = {θ}_{h} - \frac{8 π y ( t )}{λ} + 2 {Δφ}_{2} ( t ) & ( 20 )\end{matrix}$$

Mathematically, ϕ_{f}(t) and ϕₕ(t) can be extracted by using arctangent demodulation as follows:

$$\begin{matrix}{ϕ}_{f} ( t ) = arctan ( \frac{{B}_{fQ} ( t )}{{B}_{fI} ( t )} ) & ( 21 ) \\ {ϕ}_{h} ( t ) = arctan ( \frac{{B}_{hQ} ( t )}{{B}_{hI} ( t )} ) & ( 22 )\end{matrix}$$

The desired target motion x(t) is then extracted by adding 2ϕ_{f}(t) and ϕₕ(t), according to equations (15) and (20).

It should be understood that, besides the arctangent demodulation as shown in equations (21) and (22), other processing techniques, including, but not limited to, linear demodulation and complex signal demodulation, may be used to extract the information of ϕ_{f}(t) and ϕₕ(t) from equations (16)-(19).

In one embodiment, using a digital I/Q demodulation technique allows to achieve I/Q phase and amplitude balance in the receiver 206.

In one embodiment, the output signals of the receiver 206 whose frequencies are far away from DC (i.e. above a given threshold) avoid the region of highest flicker noise in the mixer output. Indeed, as understood by one skilled in the art, flicker noise has a 1/f characteristic and the higher the output frequency, the lower the flicker noise. The receiver 206 may therefore exhibit low flicker noise and high SNR.

In one embodiment, linear demodulation is used to demodulate the down-converted IF₁ and IF₂ signals. Linear demodulation is a procedure of projecting the I channel and Q channel baseband data to a single dimension through linear combination, maximizing variance in the data and suppressing redundant information. An ANC technique, which may be based on any suitable algorithm such as normalized least mean squares (NLMS), is then used to remove the unwanted radar platform motion y(t). The demodulated IF₁ signal, which corresponds to the superposition of the target motion and the platform motion, can be considered as a useful signal with noise and the demodulated IF₂ signal, which only contains the information related to the platform motion, can be considered as a reference signal. By subtracting the reference signal (i.e. the radar platform motion component) from the demodulated IF₁ signal using the ANC technique, the resulting signal contains information that is free of motion artifacts of the radar platform. Therefore, after implementation of the ANC technique, the desired target motion can be successfully extracted from the demodulated IF₁ signal. In other words, in one embodiment, using the proposed FHMF radar system (reference 100 in FIG. 1A) with ANC allows to extract vital signs of the target even in the presence of large radar platform motion.

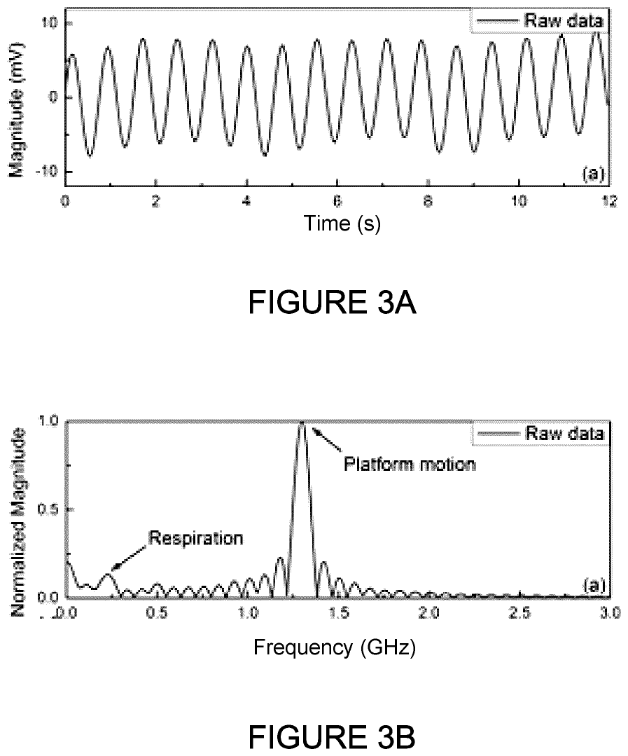

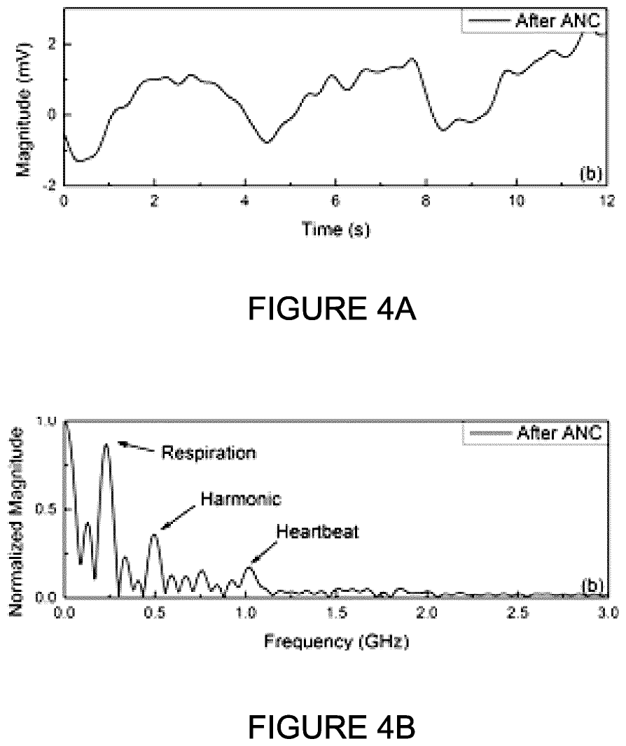

This is illustrated in FIG. 3A, FIG. 3B, FIG. 4A, and FIG. 4B. FIG. 3A and FIG. 3B respectively illustrate the measured time-domain and frequency-domain results for the demodulated IF₁ signal before ANC (labelled “raw data” in FIG. 3A and FIG. 3B). It can be seen that, because the amplitudes of the target motion are much smaller than the amplitude of the radar platform motion, it is difficult to differentiate between the target motion and the radar platform motion. As shown in FIG. 3B, the peak of the target's respiration is difficult to identify while the peak of the target's heartbeat is overwhelmed by the radar platform motion and can barely be visualized. FIG. 4A and FIG. 4B respectively illustrate the measured time-domain and frequency-domain results for the demodulated IF₁ signal after ANC. It can be seen that both the target's respiration and heartbeat can be clearly identified, and that the frequency component introduced by the radar platform motion has been effectively removed. It should be mentioned that the frequency labelled “harmonic” in FIG. 4B is the second harmonic frequency of the target's respiration.

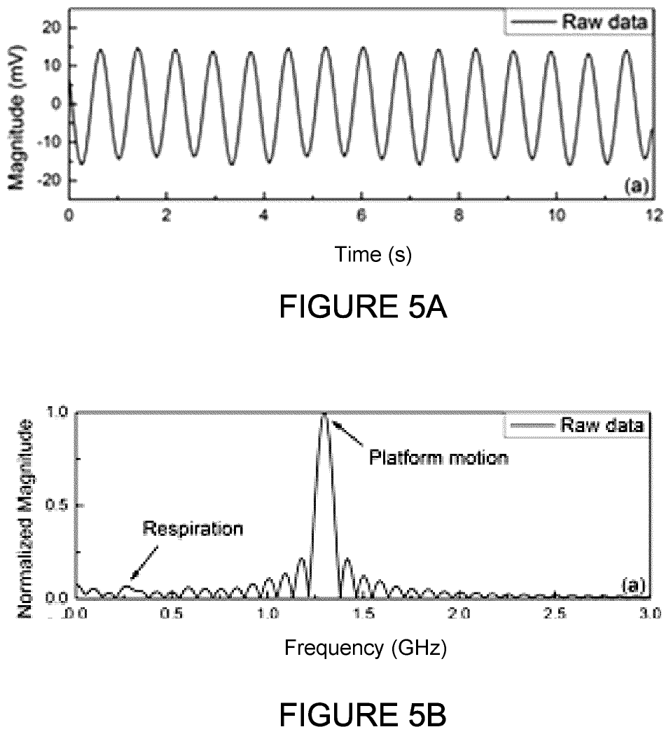

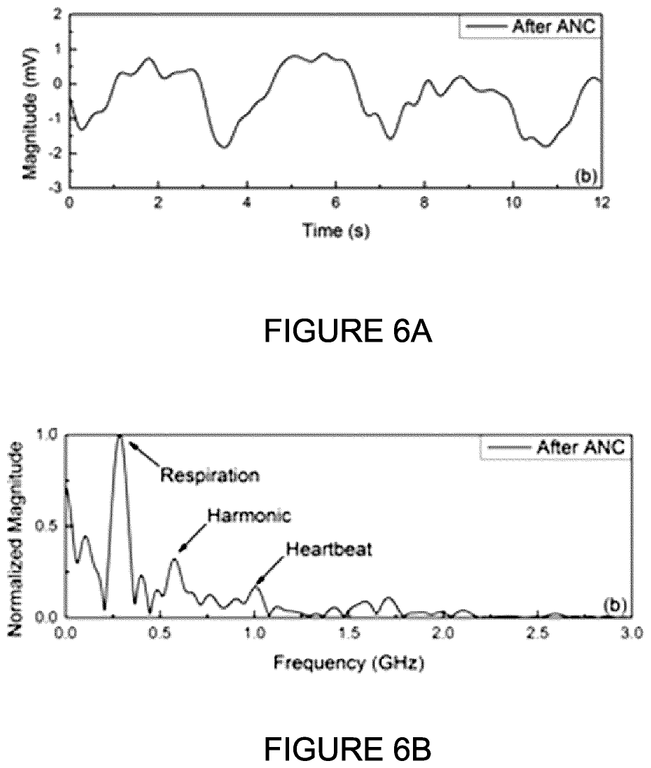

Similarly, FIG. 5A and FIG. 5B respectively illustrate the measured time-domain and frequency-domain results for the demodulated IF₁ signal before ANC (labelled “raw data” in FIG. 5A and FIG. 5B) for a STW application. It can be seen that, due to the presence of an obstacle (reference 105 in FIG. 1B), e.g. a wall, the amplitudes of the target motion are smaller than those illustrated in FIG. 3A and FIG. 3B. The target motion is therefore more difficult to identify. As shown in FIG. 5B, even the peak of the respiration is hardly seen. Nevertheless, the respiration and the heartbeat of the target can still be successfully extracted by using ANC, as shown in FIG. 6A and FIG. 6B. As such, the first set of signals may comprise a first set of frequency components of T₁*f₀, T₂*f₀, . . . Tₖ*f₀ of a periodic oscillating signal, where T₁˜Tₖ are positive integers and f₀ is the fundamental frequency of the Fourier decomposition of the signal, and the second set of signals may comprise a second set of frequency components of P₁*f₀, P₂*f₀, . . . Pₙ*f₀ of the periodic oscillating signal, where P₁˜Pₖ are positive integers.

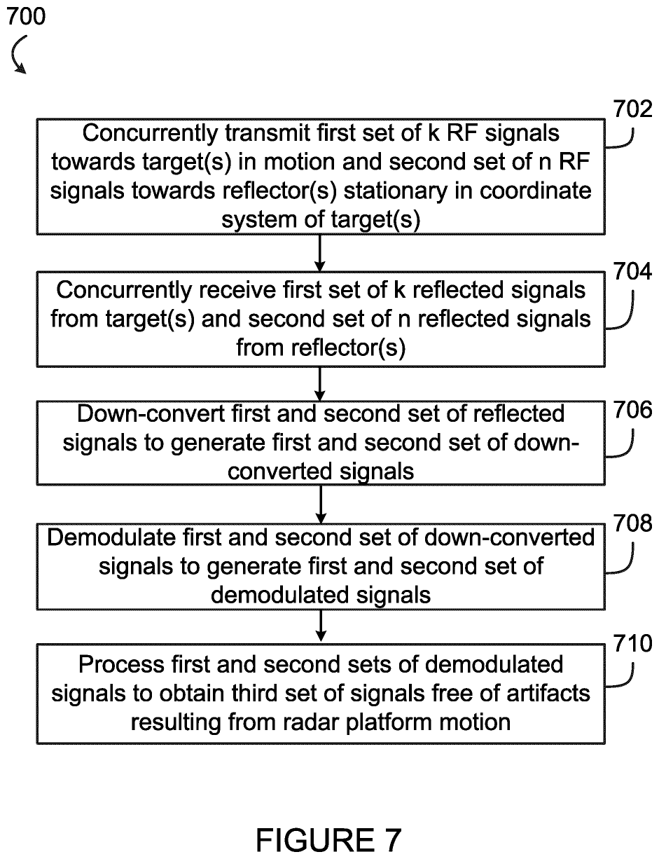

Referring now to FIG. 7, a method for operating a Doppler radar system, such as the system 100 of FIG. 1A, will now be described. The method comprises concurrently transmitting a first set of k RF signals towards one or more targets in motion and a second set of n RF signals towards one or more reflectors that is stationary in the coordinate system of the targets (step 702). The first set of k signals have a first set of k frequencies and the second set of n signals have a second set of n frequencies. In other words, step 702 may comprise concurrently transmitting 1^{st}, 2^{nd}, . . . k^{th} radio frequency (RF) signals towards one or more targets in motion and k+1^{th}, k+2^{th}, . . . k+n^{th} RF signals towards one or more RF signal reflectors, the first set of signals having first k frequencies and the second set of signals having second n frequencies.

The next step 704 is to concurrently receive a first set of k reflected signals from the one or more targets and a second set of n reflected signals from the reflectors. The first set of k reflected signals is modulated by motion of the targets and by motion of the radar platform and the second set of n reflected signals is modulated by motion of the radar platform. The first and second sets of reflected signals are then down-converted, thereby generating a first set of k down-converted signals and a second set of n down-converted signals (step 706). The first and second sets of down-converted signals are then demodulated (step 708), thereby generating a first set of k demodulated signals and a second set of n demodulated signals. At step 710, the first and second sets of demodulated signals are processed to obtain a third set of signals free of artifacts resulting from motion of the radar platform. In one embodiment, step 710 comprises subtracting the second set of demodulated signals from the first set of demodulated signals to obtain the third set of signals.

It should be noted that the present invention can be carried out as a method, can be embodied in a system or on a computer readable medium. The above description is meant to be exemplary only, and one skilled in the art will recognize that changes may be made to the embodiments described without departing from the scope of the invention disclosed. Modifications which fall within the scope of the present invention will be apparent to those skilled in the art, in light of a review of this disclosure, and such modifications are intended to fall within the appended claims.

1. A Doppler radar system comprising:
a transceiver mounted on a moving platform, the transceiver configured to:
concurrently transmit a first set of radio frequency (RF) signals and a second set of RF signals, the first set of signals having a first set of frequencies and transmitted towards one or more targets in motion, and the second set of signals having a second set of frequencies and transmitted towards one or more RF signal reflectors stationary in the coordinate system of the one or more targets,
concurrently receive a first set of reflected signals and a second set of reflected signals, the first set of reflected signals received from the one or more targets and modulated by motion of the one or more targets and by motion of the radar platform, and the second set of reflected signals received from the one or more reflectors and modulated by motion of the radar platform, and
down-convert the first set of reflected signals and the second set of reflected signals to generate a first set of down-converted signals and a second set of down-converted signals; and
a processing unit configured to demodulate the first set of down-converted signals and the second set of down-converted signals to generate a first set of demodulated signals and a second set of demodulated signals.

2. The system of claim 1, wherein the processing unit is configured to process the first and second sets of demodulated signals to obtain a third set of signals free of artifacts resulting from motion of the radar platform.

3. The system of claim 2, wherein the processing unit is configured to subtract the second set of demodulated signals from the first set of demodulated signals to obtain the third set of signals.

4. The system of claim 1, wherein the processing unit is further configured to digitize and process the first set of down-converted signals and the second set of down-converted signals prior to demodulation thereof.

5. The system of claim 1, wherein the first set of signals comprise a first set of frequency components of T₁*f₀, T₂*f₀, . . . Tₖ*f₀ of a periodic oscillating signal, where T₁˜Tₖ are positive integers and f₀ is the fundamental frequency of the Fourier decomposition of the signal, and the second set of signals comprises a second set of frequency components of P₁*f₀, P₂*f₀, . . . Pₙ*f₀ of the periodic oscillating signal, where P₁˜Pₖ are positive integers.

6. The system of claim 1, wherein the transceiver comprises a first set of antennas configured to transmit the first set of signals and receive the first set of reflected signals, and a second set of antennas configured to transmit the second set of signals and receive the second set of reflected signals.

7. The system of claim 1, wherein the transceiver comprises a first set of transmitting antennas configured to transmit the first set of signals, a first set of receiving antennas configured to receive the first set of reflected signals, a second set of transmitting antennas configured to transmit the second set of signals, and a second set of receiving antennas configured to receive the second set of reflected signals.

8. (canceled)

9. The system of claim 1, wherein the first set of signals and the second set of signals are generated by converting a sum of one or more sinusoidal signals into a sum of harmonic components.

10. The system of claim 1, wherein the first set of signals and the second set of signals are generated from a combination of a plurality of outputs of a plurality of signal generators.

11. The system of claim 1, wherein the transceiver comprises a coherent multi low-intermediate frequency (IF) receiver configured to concurrently receive the first and second sets of reflected signals, the receiver comprising multiple coherent low-IF receiver chains in parallel.

12. The system of claim 1, wherein the transceiver comprises a single set of circuitries used for concurrently transmitting the first and second sets of signals and concurrently receiving the first and second sets of reflected signals.

13. A method for operating a Doppler radar system, the method comprising:
concurrently transmitting a first set of RF signals and a second set of RF signals, the first set of signals having a first set of frequencies and transmitted towards one or more targets in motion, and the second set of signals having a second set of frequencies and transmitted towards one or more RF signal reflectors stationary in the coordinate system of the one or more targets;
concurrently receiving a first set of reflected signals and a second set of reflected signals, the first set of reflected signals received from the one or more targets and modulated by motion of the one or more targets and by motion of a moving radar platform, and the second set of reflected signals received from the one or more reflectors and modulated by motion of the radar platform;
down-converting the first set of reflected signals and the second set of reflected signals to generate a first set of down-converted signals and a second set of down-converted signals;
demodulating the first set of down-converted signals and the second set of down-converted signals to generate a first set of demodulated signals and a second set of demodulated signals, and
processing the first and second sets of demodulated signals to obtain a third set of signals free of artifacts resulting from motion of the radar platform.

14. The method of claim 13, wherein processing the first and second sets of demodulated signals comprises subtracting the second set of demodulated signals from the first set of demodulated signals to obtain the third set of signals.

15. The method of claim 13, further comprising digitizing and processing the first set of down-converted signals and the second set of down-converted signals prior to demodulation thereof.

16. The method of claim 13, wherein concurrently transmitting the first set of RF signals and the second set of RF signals comprises concurrently transmitting the first set of signals comprising a first set of frequency components of a periodic oscillating signal and the second set of signals comprising n a second set of frequency components of the periodic oscillating signal.

17. The method of claim 13, wherein the first set of signals and the second set of signals are generated by converting a sum of one or more sinusoidal signals into a sum of harmonic components.

18. The method of claim 13, wherein the first set of signals and the second set of signals are generated from a combination of a plurality of outputs of a plurality of signal generators.

19. The method of claim 13, wherein the first and second sets of reflected signals are concurrently received at a coherent multi low-intermediate frequency (IF) receiver comprising multiple coherent low-IF receiver chains in parallel.

20. The method of claim 13, wherein the first and second sets of signals are concurrently transmitted, and the first and second sets of reflected signals are concurrently received via a single set of circuitries.

21. A receiver for a Doppler radar system, the receiver comprising:
a first set of receiving antennas operating at a first set of frequencies and configured to receive a first set of RF signals, the first set of signals reflected from one or more targets in motion and having the first set of frequencies;
a second set of receiving antennas operating at a second set of frequencies and configured to receive a second set of RF signals, the second set of signals reflected from one or more reflectors stationary in the coordinate system of the one or more targets, the second set of signals having the second set of frequencies;
a first set of output ports and a second set of output ports;
a first signal channel connected to the first set of receiving antennas and the first set of output ports and a second signal channel connected to the second set of receiving antennas and the second set of output ports, the first and second signal channels each comprising one or more signal paths; and
pseudo-diplexer circuitries configured to selectively direct the first set of reflected signals over the first signal channel and the second set of reflected signals over the second signal channel.
